(19) United States
(12) Patent Application Publication
(10) Pub. No.: US 2009/0286317 A1
(43) Pub. Date: Nov. 19, 2009

(54) MODULAR CULTURE SYSTEM FOR MAINTENANCE, DIFFERENTIATION AND PROLIFERATION OF CELLS

(75) Inventors: Christian Demmler, Berlin (DE); Christoph Giese, Berlin (DE); Richard Ammer, Berlin (DE); Uwe Marx, Berlin (DE)

Correspondence Address: FOLEY AND LARDNER LLP;SUITE 500, 3000 K STREET NW, WASHINGTON, DC, 20007, US

(73) Assignee: PROBIOGEN AG

(21) Appl. No.: 12/310,446

(22) PCT Filed: Sep. 13, 2007
(86) PCT No.: PCT/EP2007/059676

§ 371 Date: Jun. 26, 2009

Related U.S. Application Data
(60) Provisional application No. 60/825,585, filed on Sep. 14, 2006.

(51) Int. Cl. C12N 5/00; C12M 1/00

(52) U.S. Cl. 435/377; 435/289.1; 435/303.1; 435/325

(57) ABSTRACT

The invention provides a culture system, which combines a device for undifferentiated proliferation of the cells with a device for cell differentiation and proliferation. Aspects of the invention include a modular culture system, a kit and an associated method for combining undifferentiated proliferation of cells with differentiation and proliferation of cells in a single, integrated device. The culture system has a first compartment comprising a plurality of miniaturized culture cavities that are sealed on one end by a surface that may be perforated, and a second compartment which defines one or more, larger culture chambers, and wherein said first and second compartment can be sterilely connected and living cell material can be transferred directly and in a controlled manner from the first compartment into the second compartment.

### FIELD OF THE INVENTION

The invention relates to a culture system, a kit and a method for undifferentiated proliferation, maintenance, differentiation and proliferation of cells.

### BACKGROUND OF THE INVENTION

Recent discoveries in stem cell technologies have encouraged research on the regeneration of human organs from embryonic or adult stem cells, aiming at finding solutions for organ and tissue repair. Tissue culture techniques require in addition to efficient oxygen and nutrient supply, the establishment of local gradients of (i) growth and differentiation factors and nutrients, (ii) oxygen tension, and (iii) pH and other (undiscovered) parameters, as well as structured surfaces for chemotaxis and local settlement (including intercellular cross-talk through tight junctions between them), which have been described as prerequisites for the proper emulation of in vivo environments (Griffith, L. G. and Swartz, M. A. 2006. Capturing complex 3D tissue physiology in vitro. Nat Reviews Molecular Cell Biology, 7: 211-224). For this purpose, heterogeneous culture systems need to be developed with particular emphasis on controlled, continuously adjustable, long-term culture processes. The basic aims of these cell culture device and process developments are to create an architecture and homeostasis mimicking the relevant human microenvironment for self-organisation of a specific tissue. For example, U.S. Pat. No. 5,516,691 relates to a cell culture module, which provides for material exchange between microorganisms/cells. Thereby, the conditions of physiological organs can be simulated, such that at virtually any point of a close packed network a few microorganisms have almost identical conditions for substrate supply. U.S. Pat. No. 6,306,644 and U.S. Pat. No. 6,255,106 describe a cell culture process and device for simulating organic interactions on the humoral plane.

With the recent discoveries in human stem cell research, substantial knowledge has been acquired about how stem cells self-renew and produce differentiated progeny under homeostatic conditions during ontogeny (Okazaki, K. M. and Maltepe, E. 2006. Oxygen, epigenetics and stem cell fate. Regenerative Med. 1: 71-83) and in adults. With these latest findings different devices and methods for stem cell proliferation appeared (U.S. Pat. No. 6,326,198, 20030017589/US-A1, 20050233448/US-A1).

All the above-mentioned culture systems can be divided into two groups, i.e., culture systems supporting cell differentiation and proliferation (U.S. Pat. No. 5,932,459, U.S. Pat. No. 6,972,195) and culture systems providing a microenvironment for proper undifferentiated proliferation (U.S. Pat. No. 5,670,351, 20050233448/US-A1). Applying particular methods, in vitro proliferation and trans-differentiation could be achieved (20040127406/US A1). However, none of the systems currently available permits both functions within a single modular device with a specific kit and method to transfer proliferated cells into a maintenance and differentiation device. Surprisingly, the present invention fulfills this and related needs.

### SUMMARY OF THE INVENTION

The invention provides a culture system, which combines a device for undifferentiated proliferation of the cells with a device for cell differentiation and proliferation. Aspects of the invention include a modular culture system, a kit and an associated method for combining undifferentiated proliferation of cells with differentiation and proliferation of cells in a single, integrated device.

According to the invention, a modular culture system is understood to mean a sterile cell culture apparatus that has a first compartment comprising a plurality of miniaturized culture cavities and wherein the cavities are sealed on one end by a surface that may be perforated, and a second compartment which defines one or more, larger culture chambers, and wherein said first and second compartment can be sterilely connected and living cell material can be transferred directly and in a controlled manner from the first compartment into the second compartment.

This culture system allows mimicking undifferentiated proliferation, differentiation and proliferation processes and maintenance of cellular material and tissues, as they appear in respective tissue niches in the body. The modular system combines a cell proliferation device under maintenance of stem cell potency (first compartment) with a differentiation and proliferation device (second compartment). The first compartment comprises a plurality of miniaturized culture cavities, which allow cultivation of living cell material with comparable culture conditions (parallel culture). The second compartment may optionally also comprise more than one culture chamber for parallel cultures. In particular, the first and second compartments may be incubated under different culture conditions, which are defined by culture media, supplements, matrices, technically supported microenvironment and gas supply.

In one embodiment of the invention, the first and second compartments can be sterilely connected on top of each other and cells, tissues and organoids can be vertically transferred directly and in a controlled manner from the upper into the lower compartment. In another embodiment, the first and second compartments are sterilely connected on top of each other.

The culture system of the invention allows the use of a variety of culture conditions. The culture in the second compartment is independently selected from the group consisting of batch culture and a culture with defined, controlled and continuous or periodic exchange of cell culture media and supplements.

In one embodiment, living cell material is present in the first compartment or, after transfer, in the second compartment. In another special embodiment, different cells can be cultivated in the first and second compartment simultaneously. After transferring cells from the first compartment on top of a matrix assisted culture in the second compartment, a dual layer structure containing at least two cell fractions can form.

Different types of living cell material such as cells, tissues and organoids can be cultured in this system. The culture device is not limited to adherent cells since cells can be kept in suspension using semi-solid or gel matrices (e.g. methyl celluloses, fibrin gel, collagen gel Matrigel™/BD Biosciences). The culture system is made from non-cytotoxic, cell culture-tested material, such as polypropylene (PP), polystyrene (PS), polyoxymethylene (POM), polysulfone, polyethersulfone (PES), polyetheretherketone (PEEK) or polytetrafluoroethylene (PTFE).

In a further aspect, the invention provides a method of setting up a two-stage culture system for cultivation of cells, tissues or organoids comprising the following steps:

a) cultivating living cell material in a first compartment comprising a plurality of culture cavities of miniaturized culture volume that are sealed on one end by a surface that may be perforated,

b) sterilely connecting the culture cavities of the first compartment with a second compartment with an enlarged culture volume, and

c) sterilely transferring living cell material cultured in the cavities from the first compartment into the second compartment.

The invention also provides a kit comprising a modular culture system and a sterile transfer device.

A direct and controlled transfer greatly minimizes the risk of contamination and increases biosafety, which is of outmost importance. In particular, if the cultivated living cell material is to be used for further analytical or even preparative purposes, such as e.g., as an implant for a patient. Controlled transfer means depositing cells from selected cavities of the first compartment onto certain selected culture surfaces (e.g. glass slides, matrices or matrix assisted cell cultures) of the second compartment. The modular cell culture system of the invention obviates the need for laborious and time consuming cell splitting and feeding operations, and thereby allows to significantly decrease production times and costs.

### BRIEF DESCRIPTION OF THE FIGURES

FIG. 1 shows a preferred embodiment of the device according to the invention.

FIG. 1A illustrates the cross-section through the device along axis A of the miniaturized cell maintenance and proliferation device (the first compartment) and the proliferation and differentiation device (the second compartment).

FIG. 1B shows the top-view of the first compartment and

FIG. 1C the top-view of second compartment.

FIG. 2 shows the setup for the second compartment: (A) for continuous perfusion, (B) for culturing the cells in direct contact with a gas phase, (C) for transfusion.

FIGS. 3 and 4: Microscopic analysis of A549 cells cultivated in the first (FIG. 3) and second compartment (FIG. 4).

FIG. 5: Microscopic analysis of HACAT cells cultivated in the first (top row) and second compartment (bottom row).

FIG. 6: Microscopic analysis of normal human epidermal keratinocytes (NHEK) cultivated in collagen gel in the first compartment and human hair follicle fibroblasts (hHFF) cultivated in fibrin gel in the second compartment. After transferring a NHEK cell suspension from the first compartment to the second compartment, a dual layer skin equivalent with a NHEK monolayer on top of fibrin gel containing proliferating hHFF was achieved.

### DETAILED DESCRIPTION OF THE INVENTION

### Definitions

Autocrine factors are all those substances secreted by cells, which support and mediate maintenance, growth or differentiation of the same cell that secreted the factor.

Paracrine factors are all those substances secreted by cells, which support and mediate maintenance, growth or differentiation of another but adjacent cell.

Self-conditioning describes all factors leading to improved cell behaviour.

Differentiation means the development of tissue specific functions of cultured cells.

Maintenance describes the ability to keep all functions of a given tissue constant within a given cell culture process.

Living cell material describes cells, tissues and organoids or cell aggregates.

Cells means cell lines or primary cells of vertebrates or invertebrates.

Tissue stands for biopsy material or explants taken from patients or animals.

Organoids means artificial, de novo generated, functional cell aggregates of different types of cells in vitro that show organ or tissue function.

Media stands for liquids with nutrients and substances necessary for cultivation of cells.

Supplements describe substances to be added to culture media in order to induce or modify cell function (e.g. cytokines, growth factors, serum).

Matrix describes substances or mixtures for surface coating or voluminous application to optimize cell attachment or allow 3D embedded culture. Matrix enhances proliferation, differentiation, function or tissue formation of cells. Matrices can include artificial or biogenic substances like hydrogels, foams, fabrics or non-woven fibres. Matrices are defined by structure, chemical composition and/or functionalisation, e.g., with extracellular matrix proteins.

Micro environment means local concentration of substances surrounding and influencing cells on a micrometer scale.

Batch culture describes a culture process without media exchange.

Perfusion means continuous, lateral directed media and/or gas transport.

Transfusion means continuous, vertical directed media and/or gas transport.

Growth and differentiation factors are substances released by cells, which induce proliferation (growth factor) or differentiation (differentiation factor) in other cells (paracrine factors) or in the same cell (autocrine factors). These factors can be supplemented to the cell culture media if known.

Proliferation means increase in cell mass by repeated rounds of cell division.

In one aspect, the invention provides a modular culture system comprising

a first compartment comprising a plurality of miniaturized culture cavities and

wherein the cavities are sealed on one end by a surface that may be perforated, and

a second compartment that defines one or more, larger culture chambers, wherein said first and second compartment can be sterilely connected and living cell material can be transferred directly and in a controlled manner from the first compartment into the second compartment.

The modular structure of the system offers the advantage of minimizing labour for feeding and splitting cell cultures. The modular system combines a miniaturized cell proliferation device for maintenance of stem cell potency (first compartment) with a differentiation and proliferation device (second compartment).

In a preferred embodiment, the perforable surface is a gas permeable base, which has been described to be particularly useful for cultivating embryonic stem cells (2005023448/US-A-1).

In a preferred embodiment, the miniaturized culture cavities of the first compartment are multiple culture cavities with comparable culture conditions, e.g., parallel culture conditions known to the skilled person. Similarly, the second compartment may optionally comprise more than one larger culture chamber for parallel cultures.

Different kinds of living cell material, such as cells, tissues and organoids, may be cultured in the modular culture system of the invention. Biopsy material or explants can be taken from, e.g., liver, kidney, skin or embryonic material.

The modular culture system of the invention permits that the first and second compartments can be incubated under different culture conditions. Culture conditions are defined by culture media, supplements, matrices, technically supported microenvironment and gas supply (e.g. gas-mix with 20% oxygen and 5% carbon dioxide) and may be independently selected from this group. The type of culture in the first compartment may be independently selected from the group consisting of batch culture and culture with defined and controlled exchange of cell culture media and supplements. The type of culture in the second compartment may be independently selected from the group consisting of batch culture and a culture with defined, controlled and continuous or periodic exchange of cell culture media and supplements. Technically supported microenvironment is largely influenced by media flow-rate and supplements, the inner space of the culture cavities, the type and construction of scaffold/support structures and/or matrices used for growing of living cell material.

Living cell material may be seeded in the first compartment in miniaturized culture cavities for proliferation of a population of undifferentiated cells. Living cell material may be transferred into the culture cavities by manual or automated pipetting of cell suspensions into each of the separate culture cavities, or by manual or automated deposition of biopsy material (e.g., using tweezers). Preferably, the first compartment has an inner culture volume of 20-1000 μL or 50 to 500 μl, and more preferably 50 to 300 μl. Thereby, the culture volume permits self-conditioning of the cells with autocrine factors by the cells cultivated. The person skilled in the art will be able to adjust the dimensions and shapes of the cavities to suit a particular application, while still providing adequate O₂-transfer and nutrient supply. In this miniaturized system, a low number of cells may be sufficient for inoculation (ranging from 10 to 10.000 cells), but higher numbers (up to 500.000 cells) can also be used. The first compartment comprises at least one cavity, preferably the number of cavities in the first compartment is in the range of 6 to 96, such as, e.g., 6, 12, 24, 48, 60 or 96 cavities. In a preferred embodiment cavities are arranged in an array suitable for automatization of procedures, such as pipetting of culture media and transfer of living cell material into the second compartment. Preferably, the miniaturized culture cavities are in standardized microtiter plate format for possible automatization.

In one embodiment of the invention, the first compartment is covered by a lid or foil for sterile cultivation in a cell culture incubator. The first compartment can be adapted to different culture conditions and allows for maintenance or proliferation of cells in an undifferentiated state.

Living cell material in the culture cavities is selected from the group consisting of cells, single cells, cell clusters, tissue biopsies (e.g., liver, kidney, skin or embryonic material) or organoids.

The cavities in the first compartment may contain liquid media, supplements and/or matrices for culturing living cell material. Matrices such as hydrogels (e.g. collagen gel, fibrin gel), semisolid matrices (e.g. methyl celluloses), hydrogel shredder (e.g. alginate, agarose), foams (e.g. collagen foams) or other kinds of matrices (e.g. collagen coated Cytodex 3 beads/GE Healthcare or degradable GCS Microcarrier/Global Cell Solutions), which improve self-conditioning of the cells, may be used.

Matrix describes substances or mixtures for surface coating or voluminous application to optimize cell attachment or allow 3D embedded culture. An optimal matrix would promote cell proliferation, differentiation, function and/or tissue formation of cells, expression of cell-specific phenotypes and the activity of the cells. Matrices can include artificial or biogenic substances like hydrogels, sponges, foams, fabrics or non-woven fibres. The matrix may be selected from semi-solid matrices (e.g. methyl celluloses), hydrogels (e.g. collagen gels, fibrin gel, agarose, alginate), hydrogel shredder, sponges (e.g. collagen sponges), foams (e.g. polyethersulfone (PES)-foam, polystyrene (PS)-foam), fabrics or non-woven fibres (e.g. polyamide fabrics or fibres). Other kinds of matrices, such as collagen-coated Cytodex 3 beads (GE Healthcare) or degradable GCS Microcarrier (Global Cell Solutions) may also be used. Matrices are defined by structure, chemical composition and/or functionalisation, e.g., with extracellular matrix proteins. The structure of the matrix may allow optimal transfer of nutrients, supplements and gas to the cells.

Matrices may be formed from any suitable polymer known to the person of skill in the art. The polymer is biocompatible, either biodegradable or non-biodegradable. Acceptable polymers include agarose, collagen, fibrin, alginate, hyaluronic acid, chitosan, chitin, polytrimethylene carbonate, poly hydroxybutyrate, amino acid based polycarbonates, poly vinylchloride, polyvinyl alcohol, polymethacrylate, poly fumarate, polyHEMA, polystyrene, PTFE, polyethylene glycol, or polyethylene glycol based polymers and derivatives thereof. Biodegradable polymers include polylactides, glycolides, caprolactones, orthoesters and copolymers thereof.

For example, a hydrogel may be prepared using Matrigel™. Sponges may be out of collagen (OptiMaix™ form Matricel). Foams for example may be made of polyethersulfone (GKSS) or polystyrene (Wilden AG), Non-woven fibres may be made of polyamide as used for preparation of erythrocyte concentrates for blood transfusion (Asahi) or manufactured using electro-spinning technology (J. H. Wenndorff).

The matrix may be solidified inside the culture cavity using matrix specific protocols. Living cell material may be embedded in the matrix by preparing a suspension of cells in an aqueous matrix-forming composition and solidifying the suspension. Hydrogel cell suspensions can either be solidified by decreasing the temperature (agarose) or rising the temperature of the matrix cell suspension to 37° C. (e.g. Matrigel™, Collagen, Fibrin).

Fibrin gel cultures may be prepared by suspending living cell material in an aqueous matrix forming composition comprising culture media supplemented with fibrinogen (e.g. 2.8 μg/ml), aprotinine (e.g. 25 μg/ml) and thrombin (e.g. 1.25 U/ml) and gelling the suspension by incubation at 37° C. for an effective period of time, e.g. 30 min. The fibrin, thrombin and if necessary aprotinine concentrations can be adapted to the desired consistency and long term stability of the gel.

The culture cavity may contain preformed solid matrices, especially foams (e.g. of polystyrene or polyethersulfone), sponges (e.g. collagen) or non-woven fibres (e.g. polyamide wool or electro-spun products).

Living cell material in the culture cavities may additionally be supported by a separated, exchangeable media reservoir such as a media hydrogel block, which is placed in physical contact with liquid in the cavities. For long-term cultures, the separate hydrogel block or exhausted liquid media may be replaced by a new one. In one embodiment of this invention, the hydrogel block is formed using liquid media containing agarose (e.g., Agarose type VII/Sigma) using a specially designed mould that exactly fits the inner dimensions and distance between the cavities of the first compartment.

In one embodiment, the living cell material can be cultured in the first compartment over the period of days to weeks simply by exchanging the hydrogel block on top of the culture cavities.

In a further embodiment, the living cell material in the first compartment can be cryopreserved and revitalised with high viability in situ. For example, the first compartment is transferred to −80° C. with defined freezing of about 1° C./minute and subsequently stored at −150° C. in the gas phase of liquid nitrogen.

In a preferred embodiment of the invention, the first and second compartments can be sterilely connected on top of each other and cells, tissues and organoids can be vertically transferred directly and in a controlled manner from the upper compartment into the lower compartment.

In another embodiment of the invention, the first and second compartments are sterilely connected with each other.

In a preferred embodiment of the invention, the first compartment of the modular culture system is equipped by at least one foil at the bottom of the device. Preferably, said at least one foil allows visual inspection and/or gas exchange. Depending on the use, different kinds of material may be chosen. For optimal oxygen supply and gas exchange, the cavities may be sealed by a gas permeable foil (e.g., Biofoil25/Greiner Bio-One, Frickenhausen, Germany) at the bottom.

Hypoxic conditions may also be implemented by sealing the cavities at the bottom of the first compartment with non- or low-gas permeable foil (e.g., polycarbonate, polytetrafluoroethylene, or copolymers consisting of ethylene vinyl alcohol and ethylene vinyl acetate) and covering the cavities with an oxygen diffusion limiting hydrogel layer at the top. For this purpose, the hydrogel block may comprise a material selected from the group consisting of agarose, alginate, peptide gels or polyacryl. Oxygen gradients form due to oxygen consumption by the cells, given oxygen concentration and defined diffusion limitations by the hydrogel layer (thickness: 3-10 mm, material: agarose, alginate or other hydrogels, concentration for Agarose type VII: 20-50 mg/ml) from the top.

For transferring cells cultured under hypoxic conditions in the first compartment, the hydrogel layer is removed and cells are transferred using the specially designed transfer device. In the second compartment, the cells are either incubated under normal oxygen pressure (normoxic conditions) if needed for differentiation or a special gas mix is directed through the upper ports of the second compartment and cell cultures are supplied with nutrients/supplements through the hydrogel block/support structure from below (lower ports).

In one embodiment of the invention, the first compartment is equipped with two foils at the bottom of the device, wherein the first foil separates and seals the cavities and the second foil seals the compartment sterilely. For the purpose of sterilely connecting the first and the second compartments, the second foil of the first compartment may be peeled off and both devices are stuck together. The two foils may be gas- or non-gaspermeable, whatever is appropriate for the desired use. Preferably, the two foils are translucent to allow microscopic control of the culture cavities.

By microscopic control or other means including vital stains, culture cavities of the first compartment can be selected for transfer of living cell material into the second compartment.

A cannula, which is specially designed according to the invention and connected to a syringe, may be used for the controlled and sterile transfer of the culture volume of the first compartment into the second compartment. The transfer may also be automated by using a pipetting robot equipped with a special transfer tool. The cannula or transfer tool according to the invention has an optimized cut that allows the perforation of the foil that seals the bottom of each well, preferably not punching the whole foil off, so that it is not transferred into the second compartment. The dimensions of the cannula are adapted to shape and dimensions of the cavities of the first compartment. By way of example, a sterile needle may be used to transfer expanded cells from a single cavity of the first compartment into a culture area of the second compartment for further differentiation, proliferation or maintenance. Living cell material is pushed into the needle, the flexible culture foil is perforated with the cut of the needle and the material is transferred into the second compartment by pressure.

In another embodiment the matrix containing cells in the first compartment may be digested by appropriate enzymes before transferring the living cell material, resulting in a cell or cell cluster suspension. For example, agarose gels can be digested using agarase, collagen matrices can be digested using collagenases and fibrin gels can be digested using plasmin. If the living cell material is growing in or on a non-degradable matrix, cells can be detached in the first compartment before transfer, using trypsin, accutase, alfazyme or other enzymes.

According to the invention, the second compartment has a preferred cell culture surface of 12.5 to 300 cm², preferably 25 to 100 cm², and most preferably, of approximately 60 cm². Using an insertable frame system, the culture space of the second device may be compartmentalized, e.g., for parallel cultures, with smallest compartments of approximately 3 cm² surface area. The culture volume in the second compartment is at least 10 to 100 ml, more preferably 20 to 60 ml. The second compartment may be compartmentalized to comprise 1 to 48, preferably 2 to 24 chambers. The frame in the second compartment may be manufactured such as to allow the transfer of living cell material of one or more cavities of the first compartment directly to one culture compartment in the second compartment. Living cell material can be transferred as single cells, cell clusters, organoids, or cell-matrix compounds. In case a matrix-assisted cell culture is transferred, cells can either proliferate out of the matrix or the cells are released by proteolytic digestion of the matrix (e.g. proteolysis by addition of protease or proteolytic digestion by the cells). The culture time for proteolytic digestion of the matrix is cell and matrix dependant and can range from several days up to weeks for complete disintegration of the matrix.

According to the invention, the one or more culture chambers of the second compartment can be closed by a lid or foil for further sterile cultivation in a cell culture incubator. The lid or foil may be manufactured such as to allow gas exchange with the external environment, e.g. the air in the cell culture incubator. For example the foil may be made of gas permeable Biofoil25 (manufactured by Greiner bio-one), a lid may allow gay exchange by diffusion similar to the lid of a multi-well plate.

The second compartment can be adapted to different culture conditions and allows for proliferation and differentiation of the transferred cells.

Cultivated cells, tissues and organoids sterilely transferred from the first compartment are cultured in the second compartment on a surface. Cells may also be grown on a matrix. The surface structure and matrix may be designed to provide a scaffold for 3D-embedded culture. Liquid media is supplied in a volume that assures maintenance of viability. The surface and/or matrix may be selected to allow induced differentiation and outgrowth of cells.

Alternatively, the transferred cells, tissues and organoids may be grown on glass slides. (Optionally, the glass surface may be coated with collagen, laminin, vitronectin, fibronectin, etc). Glass slides may be plate shaped with a rectangular or circular surface area and may have a surface of 79 mm², 113 mm² or 245 mm². Glass slides may be inserted into a frame that exactly fits the inner dimensions of the second compartment. For culturing cells on glass slides on the second compartment, the frame in the second compartment may contain holes with dimensions according to the used glass slides (e.g. diameter of 5, 6 or 9 mm) and hubs for inserting the glass slides. The frame may be made of polystyrene, polyethylene, polypropylene, polyetheretherketone, polysulfone, polyethersulfone, polytetrafluorethylene or polyoxymethylene. The holes in the frame are positioned such as to allow transfer of cell material from one or more cavities of the first compartment onto a glass slide of the second compartment.

In one embodiment, the living cell material is grown on a hydrogel (e.g., alginate scaffold, collagen gel, fibrin gel or agarose), which may optionally be covered by a matrix film (e.g. collagen (I, II, IV) coating, fibronectin coating or laminin coating). In another embodiment the living cell material is grown on a matrix support (e.g. Matrigel™/BD Biosciences, collagen gels or Tissuecol™/Baxter). In a further embodiment cells are grown on a perfusable membrane, which permits self-conditioning of the cells. The membrane may be biodegradable, e.g., consisting of polylactide, polyglycolide, trimethylene carbonate, or compositions of these materials or other materials known to a person of skill in the art. Depending on the materials used, the membranes degrade within a certain period of time. Cells cultured on these degrading membranes can first adhere and subsequently produce more extracellular matrix proteins. The perfusable membrane may be treated with matrix substances or a matrix film (e.g., Matrigel™ film or Tissuecol™-film). After cells have been transferred into the second compartment and settled on the hydrogel, the matrix or membrane, the initial cellular outgrowth is forming a local micro-environment by releasing autocrine and paracrine factors influenced by media flow-rate and supplements. Proliferation and differentiation are influenced by these factors.

In another embodiment cells are cultivated in parallel in the first and second compartment. In this case, cells are transferred from the first compartment onto a cell-containing matrix layer in the second compartment. A dual layer composite containing at least two cell types can be achieved in this way. For example, keratinocytes or progenitor cells thereof can be cultivated in the first compartment, while fibroblasts and/or other skin related cells (e.g. langerhans cells, melanocytes, dendritic cells, endothelial cells or progenitor cells thereof) are seeded or pre-cultivated in a matrix (e.g. collagen or fibrin gel) in the second compartment. Keratinocytes or progenitor cells thereof can be cultivated in their undifferentiated state in the first compartment. Expansion of progenitor cells while retaining their undifferentiated status can also take place in the fist compartment. The culture time can vary between days to weeks, preferably 7 to 14 days. Meanwhile fibroblasts and other skin related cells and their progenitor cells can be cultivated in the second compartment within a gel or matrix for the culture time of the keratinocytes in the first compartment or preferably, for a shorter time. When the keratinocytes have reached confluence on the gel after transfer into the second compartment, the culture mode of the second compartment can be switched from submerse culture conditions to air-lift culture e.g. by lowering the media height. The culture time for submers cultivation can vary between 4 to 14 days until the keratinocyte monolayer reached confluence, preferably 7 to 10 days. The subsequent culture time for an air-lift culture can vary between 14 to 21 days until a multilayer epidermis (stratification) has formed, and cornification with fully differentiated keratinocytes has taken place. While the gel can be pre-incubated in special fibroblast stem cell media, a commercially available, special formulated keratinocyte media (e.g. Keratinocyte Growth Media 2, Promocell) should be used for the cultivation of the dual layer composite after transfer from the first compartment. A full thickness skin equivalent can be accomplished in this way.

Depending on the use, the living cell material in the second compartment may comprise more than one population of cells (a micro-organ), which may each be characterized by a specific stage of differentiation or may be of different origin, thereby mimicking the various cell populations that occur in an organ of a living organism (e.g., skin equivalents consisting of fibroblasts and epithelial cells). In one embodiment, sterilely transferred cells may be cultivated in the second compartment to generate confluent mono- or multilayer (e.g., epithelia) or larger cellular structures like aggregates, spheroids or embroid bodies.

Living cell material may be grown on a scaffold/support, which permits to induce growth into aggregates of a particular 2D- or 3D-shape. Suitable scaffold/support structure may be chosen depending on the use and are known to the person skilled in the art (see e.g., US20050084512-A1). These include hydrogels and hydrophobic or hydrophilic matrices, which may comprise natural or synthetic polymers.

Cells, tissues and organoids cultivated in the second compartment may be continuously provided with media, supplements and gas. Media, supplements and gas may be continuously supplied through at least one inlet and at least one outlet port, valves and tubing. For peripheral tubing of the device, tubings with a small inner diameter may be chosen (e.g., 1.6 mm), but for connecting to the ports a tubing with a bigger inner diameter is preferred (e.g., with an inner diameter of 3.2 mm) that allows homogeneous media distribution and reduces the risk of clogging of the ports.

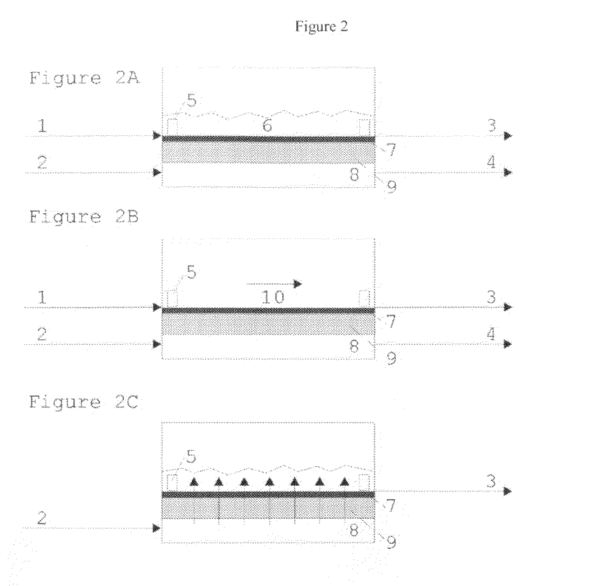

According to the invention, the cell culture in the second compartment can be transfused, perfused or cultured in direct contact with the gas phase (exemplified by the schematic drawing in FIG. 2). Culture media that enters into the second compartment may be pre-equilibrated with gas to a gas content of e.g. 20% oxygen, 5% CO₂. For this purpose, afferent ports of the second compartment may be connected with a gas permeable conduct that allows equilibrating liquid media to a pre-defined oxygen and carbon dioxide content (e.g. 20% oxygen, 5% carbon dioxide) of the environment, e.g. the air in the cell culture incubator. The gas permeable conduct may be manufactured from silicone.

Alternatively, the cell culture media may be pre-equilibrated to a predefined oxygen and carbon dioxide content with a percolator. For this purpose, the liquid reservoir of the liquid supply may be equipped with a percolator connected to an external gas supply (e.g. 20% oxygen, 5% carbon dioxide). With this embodiment, the culture system can be operated in a heating cabinet at 37° C.

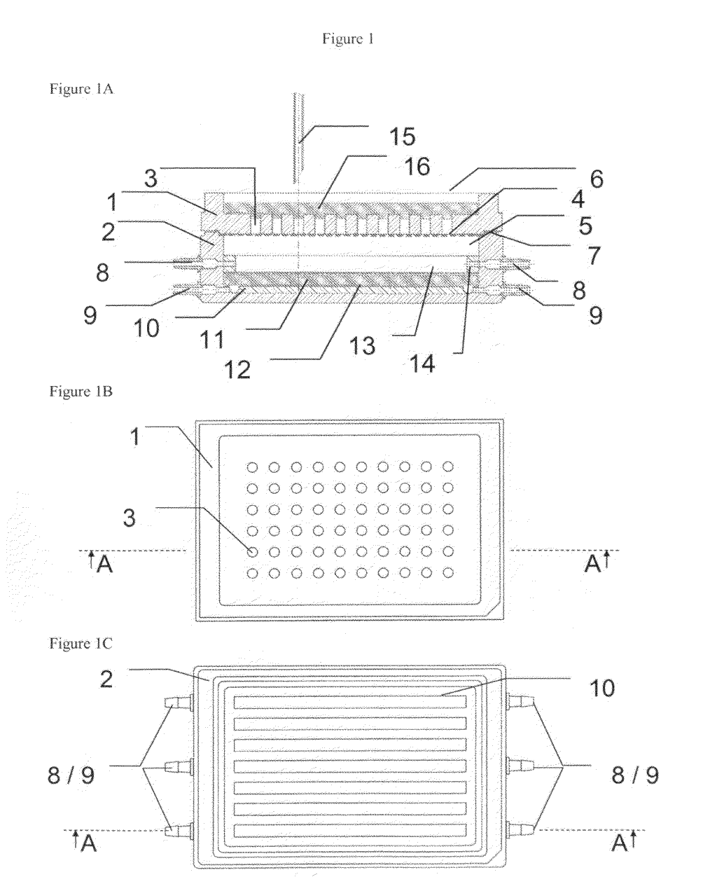

In one preferred embodiment (exemplified by the schematic drawing in FIG. 1), the second compartment is connected to two sets of afferent and efferent tubing at two different levels. The combination of port systems on two different levels allows transfusion, perfusion or direct contact of cultured living cell material with a gaseous phase. At the bottom of the culture space, the first tube set feeds into a channel system that optimizes media supply by homogeneously distributing the media under the hydrogel support. The channel system is preferably covered by a hydrogel block (e.g., agarose or alginate) and/or a perfusable membrane, optionally treated with matrix substances or a matrix-film (e.g., Matrigel™-film or Tissuecol™-film).

Transferred cells can be cultivated directly on a matrix, and optionally with matrix proteins, functionalized hydrogel block, glass slides or on a perfusable membrane (which may optionally be covered by a matrix-film).

The membrane may be mounted with a frame on top of the hydrogel surface. The frame is sitting directly on the growth surface and is manufactured to fit accurately to the dimensions of the culture compartment of the second device. The frame may also serve to compartmentalize the culture space of the second compartment. The frame can be inserted and is held in position by slight pressure from the walls.

The second compartment according to the invention offers a broad flexibility for cell feeding, waste removal and cell exposure to different conditions via a plurality of ports on two different levels. Preferably, more than one port per level is connected for supply or waste removal in order to allow homogeneous media supply. Each of the ports may optionally be closed by a (optionally vented) screw cap, when it is disconnected from the supply or waste tank. For connecting with a gas supply, the ports may optionally be supplied with a 0.2 μm membrane for consistent gas exchange and protection against contamination.

In a preferred embodiment (FIG. 1), the second compartment has twelve ports at two different levels. In particular, this supports polar culture conditions for multilayer cell cultures, by different media supplementation through lower and upper tube connections.

In one embodiment of the invention, the cells, tissues and organoids cultivated in the second compartment are cultured over the period of days to weeks. According to another embodiment of the invention, such cells, tissues and organoids cultivated in the second compartment emulate tissue- and organ functions for further analytical or preparative purposes.

The culture system, the insertable frame system and all other materials used for gas supply, media exchange, and other operations, including the transfer, are made from non-cytotoxic, sterile, (non-pyrogenic) cell culture-tested material. In one embodiment, the housing of the device is made of polypropylene, polycarbonate, polyethersulfone, polyetheretherketone, polytetrafluorethylene or polysulfone. The bottom foil may be spliced or welded to the housing. The body of the first and second compartment may be manufactured by milling and drilling. In another embodiment the first and second compartment may be produced by injection molding, notably if it is made of a thermoplastic material. Alternatively, it may be produced by compression molding, notably if it is made of a duroplastic material.

The invention furthermore provides a kit comprising a modular culture system, such as the one described above, and a sterile transfer device.

The sterile transfer device may be a cannula operated manually by a syringe or automatically by a liquid handling device. In one embodiment, the cannula is a specially ground needle. Preferably, the external diameter of the cannula exactly fits the geometry of the cavities. The cannula according to the invention allows the direct and controlled deposit of cellular material into the lower compartment. Controlled transfer may be achieved by a defined perforation of the foil that separates and seals the cavities.

Another object of the invention is to provide a method of setting up a two-stage culture system for cultivation of cells, tissues or organoids, comprising the following steps:

a) cultivating living cell material in a first compartment comprising a plurality of culture cavities of miniaturized culture volume that are sealed on one end by a surface that may be perforated,

b) sterilely connecting the first compartment with the culture cavities with a second compartment with an enlarged culture volume, and

c) sterilely transferring living cell material cultured in the cavities from the first compartment into the second compartment.

In a further aspect, step b) of the method consists of sterilely connecting the culture cavities of the first compartment with a second compartment with an enlarged culture volume by perforation of the perforable surface.

In another embodiment, the method of setting up a two-stage culture system for cultivation of cells, tissues or organoids further comprises the step of further propagating living cell material in the second compartment.

### DETAILED DESCRIPTION OF THE FIGURES

FIG. 1: Overview drawing of the first (1) and second (2) compartment on top of each other. The culture cavities (3) are sealed at the bottom by a first foil (4) and are covered by a separated, exchangeable hydrogel block (16), if applicable. The second foil (5) can be peeled off before sterile transfer of the cells from the first compartment into the second compartment. The first compartment is covered by a lid (6), that has to be removed before transferring living cell material.

A hub (7) allows the correct positioning and connection of the first (1) and the second device (2). In the second device, a hydrogel block (11) rests on a channel system (10). On top of the hydrogel block, a perfusable membrane or matrix-film (12) is fixed by a frame (14). The transferred cells are preferably cultured on this membrane (e.g., biodegradable). The culture space (13) of the second device is supplied with cell culture media and/or gas via two afferent and efferent port systems (8 and 9) preferably consisting of more than one port per level in order to allow homogeneous media and/or gas supply. The lower media ports (9) end into a channel system, which allows media distribution under the hydrogel block. Through the upper ports (8), media or gas can be directed. The combination of an upper (8) and a lower port system allows transfusion, perfusion or direct contact of cultured cellular material with the gas phase. The transfer cannula (15) indicates a cavity in the first compartment being transferred into the second compartment.

Legend FIG. 1

(1) first compartment

(2) second compartment

(3) miniaturized culture cavities

(4) first foil, sealing the culture cavities (3)

(5) second foil, serving as a sterile barrier

(6) lid to cover the first compartment

(7) hub for connecting the first and the second compartment

(8) upper ports for media or gas supply

(9) lower ports for media supply

(10) channel system for distribution of media and rest for the hydrogel block (11)

(11) hydrogel block, potentially functionalized with extracellular matrix proteins

(12) perfusable membrane or matrix layer, serving as a surface for culturing cell material

(13) culture volume, optionally filled with media

(14) insertable frame for fixation of membrane (11), potentially dividing the culture surface into different areas

(15) cannula for transferring living cell material

(16) hydrogel block serving as a separated, exchangeable media reservoir or as an impermeable barrier for implementing hypoxic conditions

FIG. 2: Schematic drawing of possible setups of the second compartment.

FIG. 2A shows the setup for continuous perfusion of the culture chamber, wherein 1 are the upper afferent and 2 are the lower afferent tubing ports. The lower tubing ports end into the channel system (8) under the hydrogel block (9), which is covered by a matrix film or perfusable, optionally with extracellular matrix proteins treated membrane (7). The perfusable membrane (7) is fixed by a frame (5), which is located at a short distance from the ports for the tubing. In this embodiment, the culture area is covered by liquid media (6). The upper und lower efferent ports have the numbers 3 und 4, respectively. Homogeneous culture conditions can be obtained by supplying the same media through the upper and lower media ports, while heterogeneous or polar media supply can be realised by guiding media A through the upper ports (1 and 3) und media B through the lower ports (2 and 4).

The system FIG. 2B shows a setup for possible cultivation of cells in direct contact with the gas phase. Through the upper afferent port (1) gas is directed through the culture area. The cells on the matrix film or perfusable membrane (7) are in direct contact with a constant gas flow (10). The gas exhausts in this embodiment through the upper efferent ports (3). The cellular material is supplied with liquid media through the hydrogel block from below by guiding media through the lower ports (2 and 4).

In a transfusion setup (FIG. 2C) the liquid media is supplied through only one afferent port system (2) comprising at least one afferent port. In this embodiment the direction of the media flow is vertical through the hydrogel block (9) and matrix film or perfusable membrane (7). Exhausted media is disposed off through at least one port for the upper efferent tubing (3).

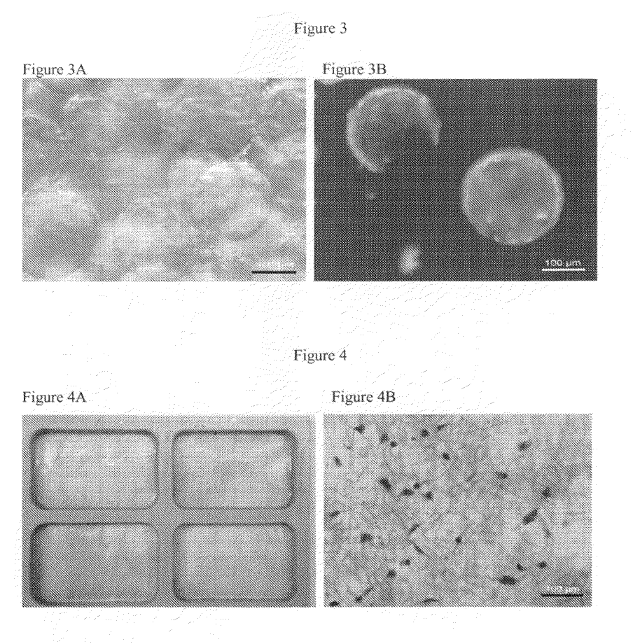

FIG. 3: FIG. 3A (left) shows the Cytodex3 carrier bead cultures of A549 cells in the first compartment. FIG. 3B (right) shows acridine orange- and ethidium bromide-stained A549 on Cytodex3 beads of the first compartment. The microscopic picture shows cells (light gray) with high vitality on the beads and suspended in the surrounding culture media.

FIG. 4:

FIG. 4A (left): Overview of the Naphtol blue-black-stained culture areas of the second compartment (A549 on a microfilamentous membrane, high cell density is indicated by darker staining of the 4 culture areas). The microscopic image of FIG. 4A on the right (4B) shows the transformed human lung epithelial cells growing on the microfilamentous membrane.

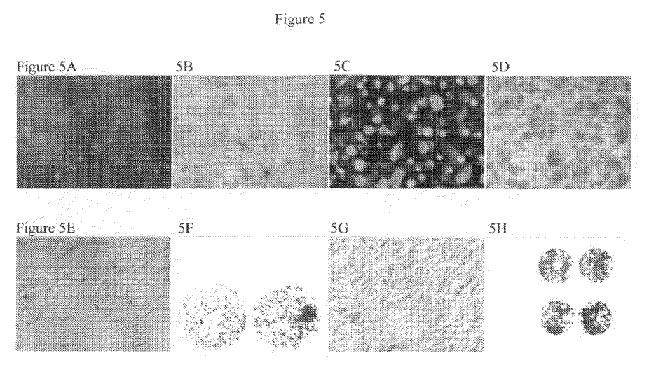

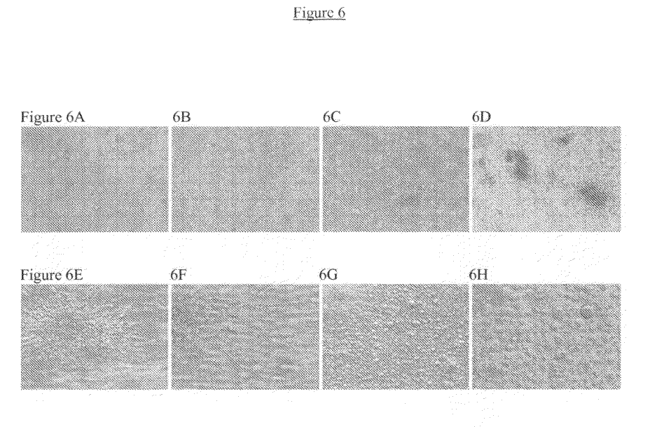

FIG. 5: P FIG. 5 (top row): Acridine orange and ethidium bromide stained cavity (5A) and phase contrast image (5B) of a cavity of the first compartment at the time point of inoculation (100× magnification) and at the time point of transfer 5 days after inoculation (100× magnification, FIGS. 5C and 5D respectively).

FIG. 5 (bottom row): Phase contrast picture (5E; 40× magnification) and image of glass slide with HACAT colonies (5F, 2 (right glass slide) and 3 (left glass slide) fibrin gel cultures transferred from first compartment) in the second compartment 7 days after transfer from the first compartment. After 7 days without perfusion, the second compartment was perfused with cell culture media for another 7 days. Phase contrast picture (5G; 40× magnification) and image of glass slide with HACAT colonies (5H, 2 (2 glass slides/top row) and 3 (2 glass slides/bottom row) fibrin cultures transferred from the first compartment) in the second compartment 14 days after transfer from the first compartment.

FIG. 6

FIG. 6 (top row): Normal human epidermal keratinocytes (NHEK) in collagen-I gel in the first compartment at inoculation (6A) and 3 days after inoculation just before transfer to the second compartment (6B) at 100× magnification. 6C shows human hair follicle fibroblasts (hHFF) in fibrin gel in the second compartment after inoculation in the second compartment at 100× magnification before the keratinocyte cell suspension was transferred. 6D shows a dual layer cell composite cultured ten days in the second compartment at 40× magnification with hHFF in the fibrin gel and NREK as a monolayer on top of the gel. The focal plane of the microscope is here within the fibrin layer.

FIG. 6 (bottom row): Dual layer skin equivalent with hHFF in the fibrin gel tantamount to the dermis and NHEK as a monolayer on top of the gel tantamount to the epidermis of the skin ten days after transfer of the NHEK from the first compartment. FIGS. 6E and 6F show fibroblasts in the fibrin gel (100× and 200× magnification, respectively). The focal plane of the microscope was set within the gel. FIGS. 6G and 6H show a monolayer of keratinocytes on top of the fibrin gel (100× and 200× magnification respectively). The focal plane of the microscope for these pictures was set to the top end of the fibrin gel layer were the keratinocytes grew.

### EXAMPLES

The invention will be further illustrated by the following non-limiting examples.

### Materials and Methods:

### Example 1

Culture media: RPMI 1640 (Invitrogen, Carlsbad, Calif.) 10% FCS (Biochrom, Berlin, Germany)

Naphtol blue-black staining solution: 0.5 g/l Naphtol blue-black (Sigma, St. Louis, Mich.), 9% (v/v) acetic acid (Roth, Karlsruhe, Germany), 8.2 g/l sodium acetate (Sigma, St. Louis, Mich.). Dissolved in double demineralised water to a final volume of 11.

Hydrogel block: 50% (v/v) 50 mg/ml Agarose type VII (Sigma, St. Louis, Mich.) in double demineralised water, 40% (v/v) double concentrated RPMI 1640 (from powder media, Cambrex Bio Science, Verviers, Belgium) and 10% FCS (Biochrom, Berlin, Germany).

### Example 2

Culture media: DMEM Glutamax-I media (Invitrogen, Carlsbad, Calif.) +10% FCS (PAA, Pasching, Austria).

Fibrin gel: cell suspension (2 E5 vital cells/ml) in 2.8 μg/ml fibrinogen (Type I-S, Sigma, St. Louis, Mich.) +25 μg/ml aprotinin (Sigma, St. Louis, Mich.) +1.25 U/ml thrombin (bovine, Sigma, St. Louise, Mich.).

### Example 3

Culture media for NHEK: Keratinocyte Growth Media 2 (Promocell, Heidelberg, Germany)

Culture media for hHFF (sc media): DMEM—Ham's F-12 mix (3:1, Invitrogen, Carlsbad, Calif.) was supplemented with 10% fetal calf serum (PAA, Pasching, Austria), adenine (180 μM, Sigma, St. Louise, Mich.), insulin (5 μg/ml, Invitrogen, Carlsbad, Calif.), hydrocortison (0.5 μg/ml, Sigma, St. Louise, Mich.), cholera toxin (0.1 nM, Sigma, St. Louise, Mich.), epidermal growth factor (10 ng/ml, Invitrogen, Carlsbad, Calif.) and penicillin/streptomycin solution (1×, Invitrogen, Carlsbad, Calif.).

Collagen-I gel: Collagen-I (rat tail) was reconstituted in 10 mM acetic acid (Merck, Germany) at a concentration of 4.17 mg/ml. 71% (v/v) collagen-I solution were mixed with 10% (v/v) 10× concentrated Hanks balanced salt solution (Sigma-Aldrich, St. Louise, Mich.) and neutralised with 0.8% (v/v) 1 M sodium hydroxide solution (Merck, Germany). All solutions were pre-cooled (4° C.). Quickly, 18% (v/v) NHEK (normal human epidermal keratinocytes, Promocell, Heidelberg, Germany) cell suspension (final concentration 9.2 E6 vital cells/ml) were mixed with the collagen-I solution and subsequently pipetted into the cavities of the first compartment.

Fibrin gel: hHFF (human hair follicle fibroblast) cell suspension (2.0 E7 vital cells/ml) were mixed with 2.8 μg/ml fibrinogen (Type I-S, Sigma, St. Louis, Mich.), 25 μg/ml aprotinin (Sigma, St. Louis, Mich.) and 1.25 U/ml thrombin (bovine, Sigma, St. Louise, Mich.).

### Abbreviations:

A549—transformed human lung epithelial cells

HACAT—transformed human epithelial cells

hHFF (human hair follicle fibroblast)

NHEK (normal human epidermal keratinocytes)

FCS—fetal calf serum

(v/v)—Volume per volume

g, mg, μg—gram, milligram, microgram

1, ml, μl—litre, millilitre, microlitre

mm—millimetre

mM, μM, M—nanomole/liter, micromole/liter, mole/liter

cm²—square centimetre

PEEK—Polyetheretherketone

PTFE—Polytetrafluoroethylene

min—minute

U—Units

° C.—degree centigrade

### Example 1

Transformed human lung epithelial cells A549 (DSMZ No. ACC 107, German Collection of Microorganisms and Cell Cultures (DSMZ) Braunschweig, Germany) were cultured in RPMI 1640 10% FCS. The first compartment was equipped with two Biofoil25 (Greiner Bio-One, Frickenhausen, Germany) layers on the bottom of the cavities. The first compartment consisted of 60 cavities with an inner volume of 70 μl each. The body of the first and second compartment was made of polysulfone. The frame of the second compartment was made of PEEK segmenting the second compartment into 4 culture areas of 13 cm². The cells in the second compartment were cultured on a perfusable fibronectin (10 μg/ml, Sigma, St. Louis, Mich.) coated microfilamentous membrane (Gore, Putzbrunn, Germany, upper side polyester filament, lower side PTFE) with an average pore size of 0.2 μm. Pharmed tubing (Saint-Gobain Performance Plastics, Charny, France) with an inner diameter of 1.6 mm was used in the peripheral tubing of the device, except for the part where the media is distributed to the 3 upper and 3 lower afferent and efferent ports of the second compartment were Pharmed tubings with an inner diameter of 3.2 mm were used for homogeneous media distribution. By using tubings with bigger inner diameters, media distribution is optimized since air bubbles in the fluidic system adhere less and do not clog single ports.

Collagen-coated Cytodex3 beads (GE Healthcare, Freiburg, Germany) were pipetted with a density of 1.0 μg/well in 38 μl RPMI 1640 10% FCS in the first compartment. Log-phase A549 were detached with TrypLE Express (Invitrogen, Carlsbad, Calif.). 30 μl cell suspension containing 1×10⁵ viable cells were seeded onto the Cytodex3 beads. The culture cavities were filled up to the top with 20 μl additional RPMI 1640 10% FCS. A hydrogel block was cast in a special mold and transferred onto the cavities for better nutrient supply of the cultures. The cells were cultured for 3 days in the first compartment. The cavities were microscopically controlled.

A hydrogel block fitting the inner dimensions of the second compartment was cast in a special mold, such that it exactly fits the inner dimensions of the second compartment and transferred into the second compartment. The second compartment was filled with 60 ml RPMI 1640 10% FCS+1× antibiotic/antimycotic solution (Cambrex Bio Science, Verviers, Belgium). The micro filamentous membrane and the frame were inserted. Before the transfer, the second foil that served as a sterile barrier was removed from the bottom of the first compartment and the first compartment was sterilely connected on top of the second compartment. The lid covering the first compartment was removed and two cavities per surface compartment were transferred from the first into the second compartment using a specially ground needle fitting the inner diameter of the cavities in the first compartment. In the first compartment, the cells grow on the beads and as single cells or small aggregates between them. The needle was equipped with a syringe containing RPMI 1640 10% FCS for transfer. The transferred cells were cultured without media perfusion for 1 day. During that period, dividing cells and cell aggregates between and attached to Cytodex3 beads adhere to the membrane and grow on the membrane in the second compartment.

After one day, the peristaltic pump was started and the second compartment was continuously perfused through the three upper and lower afferent and efferent ports with a volume flow of 13.6 μl/min RPMI 1640 10% FCS+1× antibiotic/antimycofic solution (Cambrex Bio Science, Verviers, Belgium). After 6 days, the frame and microfilamentous membrane were taken out of the reactor and stained with Naphtol blue-black solution for 30 min, fixed with 4% formaldehyde (37% formaldehyde acid free, Merck Schuchard OHG, Hohenbrunn, Germany) and 4.5% acetic acid (Roth, Karlsruhe, Germany) in PBS for 15 min and washed in tap water for another 5 min. The culture surfaces were examined under the microscope.

### Results of Example 1

The cells in the first compartment were checked microscopically after 3 days of culture. A549 cells were growing on and between the Cytodex3 beads (FIG. 3A) with high viability (FIG. 3B). The viability was checked with 1 μg/ml acridine orange (Sigma, St. Louis, Mich.) and 4 μg/ml ethidium bromide (Sigma, St. Louis, Mich.) in PBS. After 6 days of culture in the second compartment, the cells were growing with a slightly inhomogeneous distribution on the microfilamentous membrane (FIG. 4A). The cells showed high vitality as checked microscopically by Naphtol blue-black vital staining (FIG. 4B).

### Example 2

The body of the first and second compartment was made of polycarbonate. The reservoirs and cavities were realised by milling and drilling. The first compartment consisted of 18 culture cavities which were sealed by two layers of Biofoil25 (Greiner bio-one, Frickenhausen, Germany). The second compartment was equipped with a frame containing holes with a hub so that circular glass slides with 18 mm diameter can be deposited into the frame. This frame exactly fits the inner dimensions of the second compartment. The frame contained 6 holes for inserting glass slides and was made of polyetheretherketone. The device was designed such that a unit of 3 cavities of the first compartment can be transferred vertically in a controlled manner onto one corresponding glass slide in the second compartment. After peeling off the outer foil of the first compartment the special transfer device was pushed through the sealing foil of the first compartment. Thereby, the matrix assisted cell cultures of the first compartment were transferred together with cell culture media onto the corresponding glass slide in the second compartment. Up to 3 cavities were transferred vertically and in a controlled manner onto I glass slide in the frame of the second compartment.

The tubing connected to the media inlet and outlet ports (diameter 3.2 mm) of the second compartment were made of PharMed tubing (Saint-Gobain; 3.2 mm inner diameter). The tubing connected the media inlet port to a media reservoir bottle via a Ismatec IPC-N 4 peristaltic pump (1.6 mm pump tubings), and the outlet port with a waste bottle.

Proliferating log-phase HACAT cells (provided by professor Lauster, DRFZ, Berlin Germany) were cultured in standard cell culture flasks in DMEM Glutamax-I media, detached with TrypLE Express (Invitrogen, Carlsbad, Calif.). The fibrin gel with cell suspension was prepared and 50 μl/cavity were filled into the cavities of the first compartment. 18 cavities (diameter 4 mm, total volume 88 μl) were inoculated in the first compartment. The first compartment was placed for 30 min at 37° C. for gelling and subsequently 15 ml cell culture media with 35 μg/ml aprotinin were added to the culture volume on top of the fibrin gel filled cavities. Direct contact of liquid media and fibrin gel assisted cultures was assured by eliminating air bubbles with the pipette. Microscopic observation showed homogenously distributed cells (FIG. 5A). One cavity was removed after gelling and stained with 1 μg/ml acridine orange and 4 μg/ml ethidium bromide (FIG. 5B).

After 5 days in culture the second (outer) foil that served as a sterile barrier was removed from the bottom of the first compartment and the lid covering the second and first compartment was removed. Sterile glass slides were placed in a frame of the second compartment. The first compartment was sterilely connected on top of the second compartment. 3 times 2 cavities were transferred onto a glass slide in the second compartment and 3 times 3 cavities were transferred onto a glass slide in the second compartment. Cavities of the first compartment were transferred to the second compartment using a specially ground needle fitting the inner diameter of the cavities in the first compartment. The transfer device was attached to a syringe filled with DMEM Glutamax-I media.

Before transfer, one cavity was microscopically inspected (FIG. 5C) stained with 1 μg/ml acridine orange and 4 μg/ml ethidium bromide (both Sigma, St. Louise, Mich.) in PBS and showed high vitality (FIG. 5D).

After transfer, the second compartment was filled with 40 ml cell culture media and cultured in a humidified incubator without perfusion. The cultures were supplied with gas by gas exchange with air in cell culture incubator analogous to cell cultures in commonly used multi-well plates. After seven days, one glass slide with 2 and one glass slide with 3 transferred fibrin assisted cavities from the first compartment were removed and microscopically checked (FIG. 5E), fixed with 2% glutaraldehyde (Merck, Darmstadt, Germany) in PBS for 10 min and stained with 1% crytal violet (Sigma, St. Louise, Mich.) in 50% ethanol (Merck, Darmstadt, Germany) for 30 min (FIG. 5F). The peripheral fluid system was subsequently connected to the second compartment via tube clips and perfusion with DMEM Glutamax-I media was started with 150 μl/hour.

After seven days of perfused culture, again glass slides with 2 and 3 transferred cavities respectively, were removed, checked microscopically and stained with crystal violet solution. Fibrin clusters which were still present after 7 days in the second compartment had now completely disintegrated. The cells in the fibrin cultures digest the surrounding matrix by proteolysis. Once released from the hydrogel supported culture, which kept the cells in a suspension-like culture, the cells from an adherent monolayer on the glass slide and exhibit different morphology.

### Results of Example 2

After inoculation of the first compartment HACAT cells were homogniously distributed (FIG. 5B, 100× magnification, phase contrast image) and showed high vitality when stained with ethidium bromide and acridine orange (FIG. 5A, 100× magnification, narrow band blue exication). The cells in the first compartment were again checked microscopically after 5 days of culture. HACAT cells were highly viable and spatially distributed with round morphology in the fibrin gel (FIG. 5D, 100× magnification, phase contrast image). The viability was checked with 1 μg/ml acridine orange (Sigma, St. Louis, Mich.) and 4 μg/ml ethidium bromide in PBS (FIG. 5C, 100× magnification, narrow band blue exication). After 7 days of culture in the second compartment, the cells were growing in islets in typical adherent HACAT morphology (FIG. 5E, 40× magnification, phase contrast image). Glass slides were about 20% to 40% confluent, depending on the number of transferred cavities from the first compartment (FIG. 5F). After 14 days of culture in the second compartment HACAT proliferated (FIG. 5G, 40× magnification, phase contrast image) and glass slides were 70% to 80% confluent, depending on the number of transferred cavities from the first compartment (FIG. 5H). Thus, whereas cells had grown in clusters in a suspension like culture in the first compartment, epithelial HACAT cells had formed an adherent monolayer in the second compartment.

### Example 3

The body of the first and second compartment was made of polycarbonate. The reservoirs and cavities were realised by milling and drilling. The frame in the second compartment was made of polyetheretherketone. The device was designed such that one single cavity in the first compartment can be transferred vertically in a controlled manner onto one culture area in the second compartment. The frame in the second compartment exactly fits the inner dimensions of the second compartment. Holes with a hub were milled into the frame so that a glass slide with 12 mm diameter can be deposited into the frame. In this implementation, the second compartment was cultured without perfusion. The first compartment was equipped with two layers of biofoil25 at the bottom of the plate. The outer foil served as a sterile barrier, the inner foil sealed the cavities. Before transfer of cavities in the first compartment, the outer foil was peeled off and the inner foil was perforated by the special transfer device.

Proliferating log-phase NHEK cells (normal human epidermal keratinocytes, Promocell, Heidelberg, Germany) were cultured in standard cell culture flasks in Keratinocyte Growth Media 2 and detached with TrypLE Express (Invitrogen, Carlsbad, Calif.). A collagen-I gel with cell suspension was prepared and 50 μl/cavity (4.6 E5 viable cells/cavity) were added to the cavities in the first compartment. 4 cavities were inoculated in the first compartment.

The first compartment was placed for 30 min at 37° C. for gelling and subsequently 15 ml cell culture media were added to the culture volume on top of the collagen gel filled cavities. Direct contact of liquid media and collagen gel assisted cultures was assured by eliminating air bubbles with the pipette. Microscopic observation showed suspension like, homogenously distributed cells in the gel (100× magnifications, FIG. 6A).

Proliferating log-phase primary hHFF cells (human hair follicle fibroblasts), cultured in standard cell culture flasks in sc-media were detached with TrypLE Express (Invitrogen, Carlsbad, Calif.). Glass slides were placed in a frame of the second compartment. 3 days after inoculation of the first compartment, the fibrin gel with hHFF cell suspension was prepared and 300 μl/cavity were seeded onto glass slides (diameter 12 mm) in the second compartment and gelled for 15 min at 37° C. The hHFF cells were pre-incubated with sc-media (15 ml) in the second compartment for 6 hrs before the NHEK were transferred on top of this cell containing fibrin gel. One slide was microscopically inspected (FIG. 6C, 100× magnification) after inoculation but before transfer of NHEK from the first compartment.

3 days after inoculation of NHEK in the first compartment, the cavities were inspected microscopically (100× magnification, FIG. 6B) and liquid media in the first compartment was discarded. 15 mL collagenase-IV/DNAse mix (533 U/mL and 27 μg/mL respectively, Sigma, St. Louise, Mich.) were pipetted into the reservoir above the culture cavities and incubated for 2 hrs at 37° C. until the gel was disintegrated and single cells and loose clusters of NHEK cells were in the culture cavities. The collagenase-IV/DNAse mix in the reservoir was discarded using a serological pipette and the second foil of the first compartment that served as a sterile barrier was removed from the bottom of the first compartment and the lids covering the second and first compartment were removed. The first compartment was sterilely connected on top of the second compartment. 4 cavities of the first compartment were transferred, and each was transferred onto a separate hHFF fibrin gel containing culture area in the second compartment. Cavities of the first compartment were transferred to the second compartment using a specially ground needle fitting the inner diameter of the cavities in the first compartment. The transfer device was attached to a syringe filled with sc-media. When the sealing foil was perforated, about 100 to 200 μl media was pushed through the syringe, flushing the cell suspension onto the fibroblast containing fibrin gel in the second compartment.

After transfer, the second compartment was filled with additional 15 ml Keratinocyte Growth Media 2 supplemented with 70 μg/ml aprotinin (Sigma-Aldrich, St. Louise, Mich.) and cultured in a humidified incubator without perfusion. The gas supply was provided by gas exchange with air in cell culture incubator. After one day, media in the second compartment was replaced by 25 ml Keratinocyte Growth Media 2 supplemented with 35 μg/ml aprotinin (Sigma-Aldrich, St. Louise, Mich.) using a serological pipette.

After ten days of culture in the second compartment, glass slides with fibrin gel, were removed and checked microscopically (FIG. 6E to 6H).

### Results of Example 3

A dual layer skin equivalent, referring to dermis and epidermis, was obtained using this culture device and according method (FIG. 6E to 6H). In collagen-I gel seeded keratinocytes (NHEK) in the first compartment showed homogeneous distribution (FIG. 6A) and visually slight proliferation over 3 days in the first compartment (FIG. 6B). The cells are kept in suspension like culture over this period of time. Fibroblasts (hHFF) seeded in the second compartment showed round and suspension like morphology at inoculation (FIG. 6C). After enzymatic digestion of the collagen-I gel in the first compartment and transfer of single keratinocytes and clusters thereof into the second compartment, the keratinocytes proliferated in the second compartment on top of the fibroblast containing fibrin gel and formed a monolayer. Typical keratinocyte cobblestone morphology could be seen microscopically after ten days of culture in the second compartment (FIGS. 6G and 6H). The fibroblasts in the second compartment showed proliferation in clusters (FIG. 6D to 6F).

Obviously, numerous modifications and variations of the present invention are possible in light of the above teachings. It is therefore to be understood that within the scope of the appended claims, the invention may be practiced otherwise than as specifically described herein.

1. A modular culture system comprising:
a) a first compartment comprising a plurality of miniaturized culture cavities and wherein the cavities are sealed on one end by a surface that may be perforated, and
b) a second compartment that defines one or more, larger culture chambers, and
wherein the first and second compartment can be sterilely connected and living cell material can be transferred directly and in a controlled manner from the first compartment into the second compartment.

2. The modular culture system of claim 1, wherein the miniaturized culture cavities provide comparable culture conditions.

3. The modular culture system of claim 1, wherein the living cell material is selected from the group consisting of cells, tissues and organoids.

4. The modular culture system of claim 1, wherein the first and second compartments can be sterilely connected on top of each other and cells, tissues and organoids can be vertically transferred directly and in a controlled manner from the upper compartment into the lower compartment.

5. The modular culture system of claim 1, wherein the first and second compartments are sterilely connected with each other.

6. The modular culture system of claim 1, wherein the first and second compartments can be incubated under different culture conditions.

7. The modular culture system of claim 1, wherein the first and second compartments are incubated under different culture conditions.

8. The modular culture system of claim 7, wherein the culture conditions are defined by culture media, supplements, matrices, technically supported micro-environment and gas supply.

9. The modular culture system of claim 7, wherein
a) the type of culture in the first compartment is independently selected from the group consisting of batch culture and a culture with defined and controlled exchange of cell culture media and supplements, and
b) the type of culture in the second compartment is independently selected from the group consisting of batch culture and a culture with defined, controlled and continuous or periodic exchange of cell culture media and supplements.

10. The modular culture system of claim 1, wherein the first compartment has an inner culture volume of 20-1000 μL.

11. The modular culture system of claim 1, wherein living cell material in the culture cavities is supported by a separated, exchangeable media reservoir, such as a media hydrogel block.

12. The modular culture system of claim 1, wherein the cavities contain liquid media, supplements and/or matrices for culturing living cell material.

13. The modular culture system of claim 12, wherein the living cell material is cultured in the first compartment over the period of days to weeks.

14. The modular culture system of claim 11, wherein the living cell material in the first compartment is cryopreserved and revitalised with high viability in situ.

15. The modular culture system of claim 11, wherein the living cell material is selected from the group consisting of cells, single cells, cell clusters, tissue biopsies or organoids.

16. The modular culture system of claim 1, wherein the living cell material is skin related cells or skin tissue.

17. The modular culture system of claim 1, wherein the first compartment comprises keratinocytes or progenitor cells thereof.

18. The modular culture system of claim 1, wherein second compartment comprises at least one of fibroblasts, langherans cells, melanocytes, dendritic cells, endothelial cells or progenitor cells thereof.

19. The modular culture system of claim 18, wherein a skin equivalent forms in the second compartment.

20. The modular culture system of claim 1, wherein the first compartment is equipped by at least one foil at the bottom of the device.

21. The modular culture system of claim 1, wherein the first compartment is equipped by two foils at the bottom of the device, wherein the first foil separates and seals the cavities and the second foil seals the compartment sterilely.

22. The modular culture system of claim 20, wherein the at least one foil allows visual inspection and/or gas exchange.

23. The modular culture system of claim 1, wherein the one or more culture chambers of the second compartment is closed by a lid or foil.

24. The modular culture system of claim 23, wherein cultivated cells, tissues and organoids sterilely transferred from the first compartment are cultured in the second compartment on a surface/matrix and/or in liquid media in a volume with maintenance of viability.

25. The modular culture system of claim 24, wherein the surface/matrix allows induced differentiation and outgrowth of cells.

26. The modular culture system of claim 24, wherein the transferred cells are cultivated to generate confluent mono- or multilayer (e.g. epithelia/skin) or larger cellular structures like aggregates, spheroids or embroid bodies.

27. The modular culture system of claim 24, wherein cells, tissues and organoids cultivated in the second compartment are continuously provided with media, supplements and gas.

28. The modular culture system of claim 22, wherein the cell culture in the second compartment is transfused, perfused or cultured in direct contact with the gas phase.

29. The modular culture system of claim 27, wherein the cells, tissues and organoids cultivated in the second compartment are cultured over the period of days to weeks.

30. The modular culture system of any claim 27, wherein the cells, tissues and organoids cultivated in the second compartment emulate tissue- and organ functions for further analytical or preparative purposes.

31. A kit comprising the modular culture system of claim 1, and a sterile transfer device.

32. The kit of claim 26, wherein the sterile transfer device is a cannula operated manually by a syringe or automatically by a liquid handling device.

33. The kit of claim 27, wherein the external diameter of the cannula exactly fits the geometry of the cavities of the first compartment.

34. The kit of claim 27, wherein the cannula allows the controlled deposit of cellular material into the lower compartment.

35. The kit of claim 29, wherein the controlled transfer is handled by defined perforation of a foil that separates and seals the cavities.

36. A method of setting up a two-stage culture system for cultivation of cells, tissues or organoids, comprising the following steps:
a) cultivating living cell material in a first compartment comprising a plurality of culture cavities of miniaturized culture volume that are sealed on one end by a surface that may be perforated,
b) sterilely connecting the first compartment with the culture cavities with a second compartment with an enlarged culture volume, and
c) sterilely transferring living cell material cultured in the cavities from the first compartment into the second compartment.

37. The method of setting up a two-stage culture system for cultivation of cells, tissues or organoids of claim 31, further comprising the step of further propagating living cell material in the second compartment.

38. The method of claim 36, wherein the miniaturized culture cavities provide comparable culture conditions.

39. The method of claim 36, wherein the living cell material is selected from the group consisting of cells, tissues and organoids.

40. The method of claim 36, wherein the first and second compartments are sterilely connected on top of each other and cells, tissues and organoids are vertically transferred directly and in a controlled manner from the upper compartment into the lower compartment.

41. The method of claim 36, wherein the first and second compartments are incubated under different culture conditions.

42. The method of claim 36, wherein the culture conditions are defined by culture media, supplements, matrices, technically supported micro-environment and gas supply.

43. The method of claim 41, wherein
a) the type of culture in the first compartment is independently selected from the group consisting of batch culture and a culture with defined and controlled exchange of cell culture media and supplements, and
b) the type of culture in the second compartment is independently selected from the group consisting of batch culture and a culture with defined, controlled and continuous or periodic exchange of cell culture media and supplements.

44. The method of claim 36, wherein living cell material in the culture cavities is supported by a separated, exchangeable media reservoir, such as a media hydrogel block.

45. The method of claim 36, wherein the cavities contain liquid media, supplements and/or matrices for culturing living cell material.

46. The method of claim 45, wherein the living cell material is cultured in the first compartment over the period of days to weeks.

47. The method of claim 44, wherein the living cell material in the first compartment is cryopreserved and revitalised with high viability in situ.

48. The method of claim 44, wherein the living cell material is selected from the group consisting of cells, single cells, cell clusters, tissue biopsies or organoids.

49. The method of claim 36, wherein the living cell material is skin related cells or skin tissue.

50. The method of claim 36, wherein the first compartment comprises keratinocytes or progenitor cells thereof.

51. The method of claim 36, wherein second compartment comprises at least one of fibroblasts, langherans cells, melanocytes, dendritic cells, endothelial cells or progenitor cells thereof.

52. The method of claim 49, wherein a skin equivalent forms in the second compartment.

53. A method of preparing a skin tissue equivalent in a two-stage culture system comprising
a) cultivating skin related cells or progenitor cells thereof in a first compartment comprising a plurality of culture cavities of miniaturized culture volume that are sealed on one end by a surface that may be perforated,
b) cultivating at least one of fibroblasts, langerhans cells, melanocytes, dendritic cells, endothelial cells or progenitor cells thereof in a second compartment with an enlarged culture volume,
c) sterilely connecting the first compartment with the second compartment,
d) sterilely transferring the living cell material cultured in the cavities from the first compartment into the second compartment, and
e) allowing a skin equivalent to form in the second compartment.

54. The method of claim 53, wherein the skin cells in the first compartment are keratinocytes or progenitor cells thereof.

55. The method of claim 54, further comprising the step of changing the culture conditions in the second compartment when the keratinocytes have reached confluence after transfer into the second compartment.

56. A kit comprising a sterile transfer device and a modular culture system, which is characterized in that it comprises
a) a first compartment comprising a plurality of miniaturized culture cavities and wherein the cavities are sealed on one end by a surface that may be perforated, and
b) a second compartment that defines one or more, larger culture chambers.
